(19) United States
(12) Patent Application Publication
(10) Pub. No.: US 2009/0248066 A1
(43) Pub. Date: Oct. 1, 2009

(54) Elastic barbed suture and tissue support system

(76) Inventor: David Hjalmar Wilkie, Vancouver (CA)

Correspondence Address: DR. DAVID WILKIE, 8218 TIDEWATER PL., VANCOUVER, BC, V6P GRZ, CA

(21) Appl. No.: 12/078,210

(22) Filed: Mar. 28, 2008

(51) Int. Cl. A61B 17/04

(52) U.S. Cl. 606/228; 606/232

(57) ABSTRACT

A tissue support and reinforcement system uses a barbed suture capable of stretching elastically in order to allow the sutured area to act like natural tissue. This is particularly useful in cosmetic surgery.

### FIELD OF THE INVENTION

The invention relates to the medical field and is particularly useful in the field of cosmetic surgery, pelvic floor reconstruction and incontinence prevention.

### BACKGROUND OF THE INVENTION

It is a general trend in surgery to replace conventional surgery with minimally invasive surgery. In some procedure, mainly in cosmetic surgery, there is a desire to eliminate or minimize incisions. Minimizing incisions allows faster recovery, less scarring and ability to perform the procedure in a doctor's office instead of a hospital. In recent years barbed sutures such as made by Angiotech (Vancouver, BC, Canada) appeared on the market. Such sutures (shown in FIG. 1) have one-way barbs allowing easy insertion but provide significant pull-out resistance. Anchoring devices operating on similar principles and connected to conventional sutures or nets are also well known. Typically the suture has a short smooth section in its center followed by barbs pointing to the centre section of the suture. The ends are typically permanently or temporarily attached to steel needles. Both ends are deployed through the tissue and pulled till the middle is held by tissue. The tissue is then gathered against the suture to achieve the desired approximation. When sutures are tightened and cut off, they are locked in place without requiring a knot by the actions of the barbs. It was found that the system works well for some applications but can fail in areas where tissue is subject to movements and particularly stretching. Natural tissue can stretch a great amount and fully recover. When a section having a barbed suture stretches, the limited elongation of the suture causes the barbs to pull out of the tissue either by tearing the tissue or folding over of the barbs. In both cases the damage is irreversible and the holding action is lost forever. Referring now to FIG. 1, a typical prior art barbed suture 1 has barbs 2 and 2′ going in opposite directions starting from the center of the suture. The ends are typically attached to needles 5. Since cross section of barbs 2 is smaller than suture 1, the suture can not be stretched by pulling on barbs 2 and 2′. Also, sutures are made of material having limited elasticity and resilience, such as nylon or polypropylene, with a limited ability to stretch before breaking. Conventional barbed sutures can only tolerate minimal tissue stretch, in the order of 1%-2%, while the invention can easily tolerate stretches of up 100% without damage or loss of holding power.

It is an object of the invention to provide a barbed suture having an ability to stretch with the tissue without damage and then to return to normal position. It is another object to provide an elastic suture holding the tissue tight in an elastic manner, maintaining the natural look and feel of naturally elastic tissue. Sutures of this material would accommodate to normal tissue expansion that occurs as part of the inflammatory healing response to trauma or surgery with less tension and thereby less risks of hypoxic change.

### SUMMARY OF THE INVENTION

A tissue support and reinforcement system uses a barbed suture capable of stretching elastically in order to allow the sutured area to act like natural tissue. This is particularly useful in cosmetic pelvic floor repair and incontinence surgery.

### BRIEF DESCRIPTION OF THE DRAWINGS

FIG. 1 shows a prior art barbed suture.

FIG. 2 shows an elastic barbed suture based on a tightly wound coil.

FIG. 3 shows a longitudinal section of a tissue supported by an elastic barbed suture capable of stretching when the tissue is being stretched.

FIG. 4 shows an elastic barbed suture based on an elastic thread covered by rigid barbs.

FIG. 5 shows a section of a system for distributing the load across multiple elastic barbed sutures.

### DETAILED DISCLOSURE

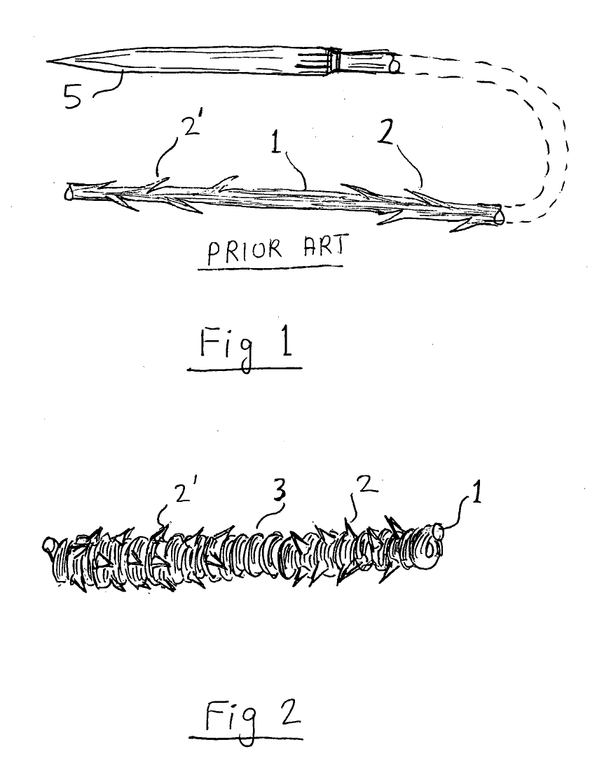

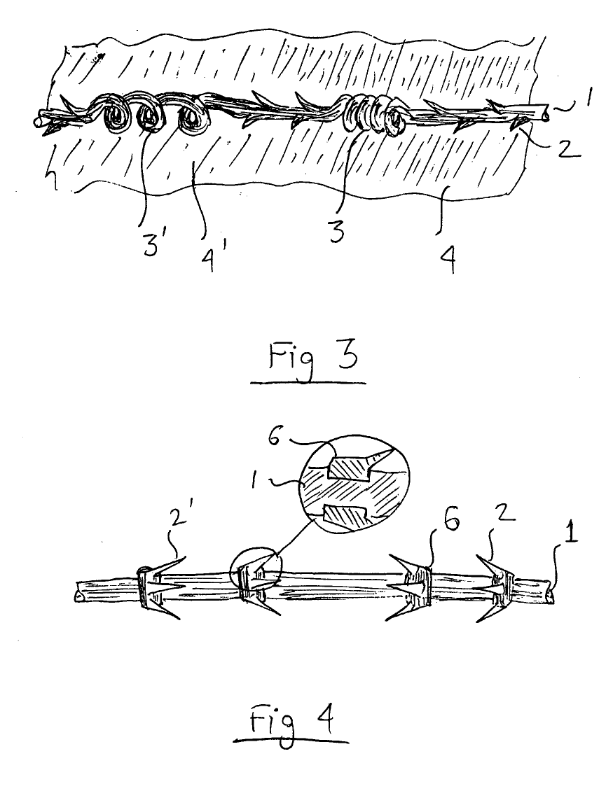

Referring now to FIG. 2, an elastic barbed suture is formed by coiling a regular barbed suture into a very tight coil 3. Barbs 2 and 2′ are oriented in a manner preventing pull-out in a direction towards the center while allowing easy deployment in a direction pointing away from the center. The complete suture may be coiled, or coiled sections 3 providing elasticity can alternate with barbed sections providing holding power, as shown in FIG. 3. In FIG. 3 tissue 4 is being supported by suture 1. When tissue 4 is stretched, as shown by area 4′, coil 3′ elongates and provides a restoring force. At the same time the holding power can never be exceeded, as coil 3 can be made slightly more elastic than tissue 4. In such a case the tissue itself will determine the maximum stretching rather than coil 3, and the holding force will never be exceeded. Such an elastic fail-safe system is particularly desirable in cosmetic surgery, such as face, breasts and vaginal surgery as well as in pelvic floor and vaginal reconstruction, and incontinence prevention surgery. The coiled section of the suture could be confined to the central section between the barbed sections, an example being only the section 3 in FIG. 2. The sutures could be placed with protruding ends in order to permit post-placement tightening adjustment. The suture ends would be removed after adjustments were complete.

Suture 1 can be made of the same materials as used for permanent (non absorbable) barbed sutures today, or can be made from absorbable materials, when only needed as temporary support, such as wound closure. The suture can also be coated or impregnated with drugs to extend its performance and can have beneficial surface finishes to increase holding force. By the way of example, it is known that a textured or dull surface increases bonding to tissue. An example of a medicated suture coating is an anti-bacterial coating. The coating can be designed for gradual release over long periods, for example by impregnating suture material with a drug. FIG. 4 shows a different embodiment of an elastic barbed suture. Suture 1 is made of an elastic material such as polyurethane or silicone rubber, or material with similar elastic properties. At fixed intervals barbed rings 6 are permanently attached to suture 1 (by use of adhesive or molding onto the suture). An advantage of this embodiment is that the size f the barb is no longer related to the diameter of the suture. This is particularly important when a large amount of tissue has to be supported, such as breast. By the way of example, suture 1 can be a 1 mm diameter polyurethane cord and barbs 2 and 2′ can be made of molded nylon and have a barb length of 2-20 mm. While the barbed ring 6 does not need to stretch, is still has to be flexible, similar to any barbed suture, to allow barbs to close while suture is inserted in tissue, and open when pulled back. Each ring can carry from 1 to over 10 barbs. The barb rings 6 can be attached by in-place molding, adhesives and by periodic grooves in elastic suture 1. In this case the suture is simply pulled through the rings, as shown in the insert drawing in FIG. 4. The pulling force exceeds the needed retention force in the tissue. As before, if suture 1 is made more elastic than the tissue, the stretching limit will be reached by the tissue before any damage to the barbed support system occurs.

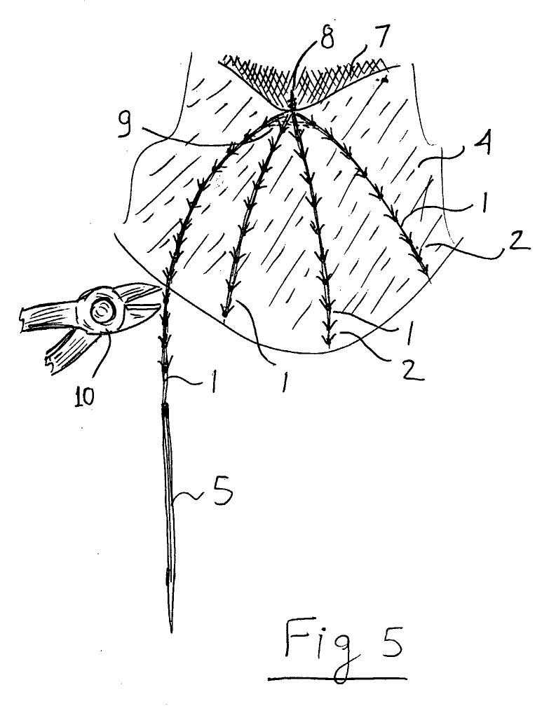

FIG. 5 shows a generic support system using the elastic barbed tissue. Tissue 4 needs to be re-shaped and supported by multiple barbed sutures 1. Sometimes a nearby ligament, bone or periosteum 7, such as pelvis, sacrum or sternum, can be used as an anchor point, using one or more conventional bone anchors 8. The screw type bone anchors can be inserted with a minimal incision or without any incision, using a large size hypodermic needle. When no rigid support is available, the center section of the suture 9 can be used as an anchor point. Multiple sutures 1 are deployed by needles 5 and cut-off below skin level. To cut suture off below skin level, the suture is temporarily stretched and then cut off flush with skin by cutter 10. The multiple sutures spread the load and maintain the elastic and resilient nature of healthy tissue. In the event that the suture must be removed, this could be facilitated by dividing the central section and withdrawing the suture halves in the same direction in which they were each inserted.

While the disclosure covers selected cases it should be interpreted broadly. The term “barbed elastic suture” should be read to apply to any support system having an elastic and resilient behavior. Such a system can be made of wires, ribbons, meshes etc. While the preferred embodiment uses a polymeric material it is obvious that a metallic barbed elastic suture can also be used. Similarly, the term “barbed rings” should be interpreted as any barbed assembly, even if it is not ring shaped. The term “elastic” implies resiliency as well. Some procedures that can benefit greatly from the use of an elastic barbed suture are: cosmetic surgery, bariatric surgery, incontinence surgery, hernia repair, sphincteric surgery, vaginal surgery and any endoscopic and laparoscopic procedure.

Where adhesion is required rather than permanent sutures, an absorbable or non-absorbable suture with adhesion forming material impregnated in or on its substance may be used.

1. A barbed suture for use in tissue and being able to accommodate said tissue stretching without damage or loss of holding power.

2. A barbed suture comprising of a highly elastic and resilient material having permanently attached flexible barbed rings.

3. A barbed suture having at least some coiled sections.

4. A tissue support system comprising of a plurality of elastic barbed sutures.

5. A barbed suture as in claim 1, 2 or 3 used in cosmetic surgery.

6. A barbed suture as in claim 1, 2 or 3 used in the treatment of incontinence, prolapse, vaginal reconstruction or vaginal rejuvenation.

7. A barbed suture as in claim 2 wherein elastic suture material is polyurethane.

8. A barbed suture as in claim 2 wherein said barbed rings are made of a flexible polymer and length of each barb is from 2 to 20 mm.

9. A barbed suture as in claim 1, 2 or 3 capable of elastically stretching by a larger amount than said tissue.

10. A barbed suture as in claim 1 for endoscopic or laparoscopic deployment.

11. A barbed suture as in claim 1 for reducing the diameter and improve sphincteric activity of an organ.

12. A barbed suture as in claims 1 for repairing hernia defects.

13. A barbed suture as in claims 1, 2 or 3 impregnated with a therapeutic substance for gradual release of said substance.
